(19) United States
(12) Patent Application Publication
(10) Pub. No.: US 2025/0161753 A1
(43) Pub. Date: May 22, 2025

(54) WORKOUT SUPPORT APPARATUS, WORKOUT SUPPORT METHOD, TRAINING APPARATUS, AND STORAGE MEDIUM

(71) Applicant: NEC Corporation, Minato-ku, Tokyo (JP)

(72) Inventor: Riki ETO, Tokyo (JP)

(73) Assignee: NEC Corporation, Minato-ku, Tokyo (JP)

(21) Appl. No.: 18/839,477

(22) PCT Filed: Mar. 30, 2022
(86) PCT No.: PCT/JP2022/015798

§ 371 Date: Aug. 19, 2024

(51) Int. Cl. A63B 24/00; A63B 71/06; G16H 20/30

(52) U.S. Cl. CPC A63B 24/0075; A63B 24/0062; A63B 71/0622; G16H 20/30; A63B 2071/0625

(57) ABSTRACT

In order to generate a workout schedule in consideration of a state regarding a workout, a workout support apparatus (2) includes: a data acquiring section (21) for acquiring state data which indicates a state regarding a workout done by a targeted person; and a generating section (22) for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

### TECHNICAL FIELD

The present invention relates to a workout support apparatus, etc. for supporting a workout.

### BACKGROUND ART

Techniques for supporting a workout has conventionally been under development. For example, Patent Literature 1 below discloses a technique of outputting a workout schedule on the basis of a look-up table and a mathematical model which are derived in advance from a statistics database on the basis of customer data or the like. Specifically, Patent

Literature 1 discloses calculating burned calories based on the exercises of a workout and the number of times a targeted person did the workout and returning a workout schedule for achieving a target value of burned calories. Patent Literature 1 also discloses returning a workout schedule having incorporated therein a psychological tendency on the basis of the result of questionnaire.

### CITATION LIST

### Patent Literature

### Patent Literature 1

Japanese Patent Application Publication, Tokukai, No. 2017-010486

### SUMMARY OF INVENTION

### Technical Problem

The technique of Patent Literature 1 is susceptible of improvement in that it is impossible to take into consideration various states regarding a workout in determining a workout schedule. For example, some of the workout exercises contained in a workout schedule generated by the technique of Patent Literature 1 may be impracticable in a workout facility used by the targeted person, or may be difficult to implement considering the physical strength of the targeted person.

As example aspect of the present invention has been made in view of the above problem, and an example object thereof is to provide a technique for generating a workout schedule in consideration of a state regarding a workout.

### Solution to Problem

A workout support apparatus in accordance with an example aspect of the present invention includes: a data acquiring means for acquiring state data which indicates a state regarding a workout done by a targeted person; and a generating means for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

A workout support method in accordance with an example aspect of the present invention includes: at least one processor acquiring state data which indicates a state regarding a workout done by a targeted person; and the at least one processor generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

A workout support program in accordance with an example aspect of the present invention causes a computer to function as: a data acquiring means for acquiring state data which indicates a state regarding a workout done by a targeted person; and a generating means for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

A training apparatus in accordance with an example aspect of the present invention includes: a data acquiring means for acquiring training data which indicates a workout schedule that is in accordance with a state regarding a workout and that is to be applied in the state; and a training means for generating an objective function for generating a workout schedule in accordance with a state, by performing inverse reinforcement learning with use of the training data.

A training method in accordance with an example aspect of the present invention includes: at least one processor acquiring training data which indicates a workout schedule that is in accordance with a state regarding a workout and that is to be applied in the state; and the at least one processor generating an objective function for generating a workout schedule in accordance with a state, by performing inverse reinforcement learning with use of the training data.

A training program in accordance with an example aspect of the present invention causes a computer to function as: a data acquiring means for acquiring training data which indicates a workout schedule that is in accordance with a state regarding a workout and that is to be applied in the state; and a training means for generating an objective function for generating a workout schedule in accordance with a state, by performing inverse reinforcement learning with use of the training data.

### Advantageous Effects of Invention

An example aspect of the present invention makes it possible to generate a workout schedule in consideration of a state regarding a workout.

### BRIEF DESCRIPTION OF DRAWINGS

FIG. 1 is a block diagram illustrating a configuration of a workout support system in accordance with a first example embodiment of the present invention.

FIG. 2 is a flowchart illustrating a flow of a training method in accordance with the first example embodiment of the present invention.

FIG. 3 is a flowchart illustrating a flow of a workout support method in accordance with the first example embodiment of the present invention.

FIG. 4 is a representation of the outline of a workout support method in accordance with a second example embodiment of the present invention.

FIG. 5 is a block diagram illustrating an example main configuration of a workout support apparatus in accordance with the second example embodiment of the present invention.

FIG. 6 is a representation of the outline of objective function training in accordance with the second example embodiment of the present invention.

FIG. 7 is a representation of an example of generating a workout schedule which contains BGM.

FIG. 8 is a representation of an example display screen with a workout schedule and BGM.

FIG. 9 is a flowchart illustrating a flow of processes carried out by the workout support apparatus in accordance with the second example embodiment of the present invention.

FIG. 10 is a diagram illustrating an example computer which executes the instructions of a program which is software for implementing the functions of each of the apparatuses in accordance with the respective example embodiments of the present invention.

### EXAMPLE EMBODIMENTS

### First Example Embodiment

The following description will discuss a first example embodiment of the present invention in detail, with reference to the drawings. The present example embodiment is basic to example embodiments described later.

### Workout Support System

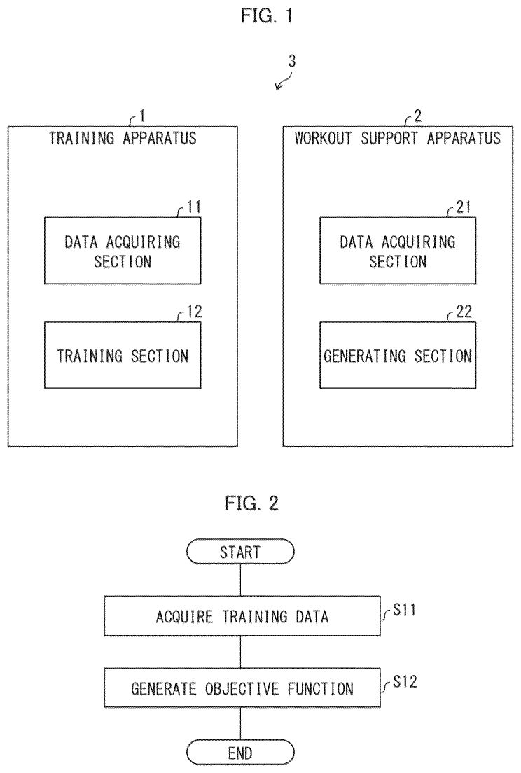

A workout support system 3 in accordance with the present example embodiment will be described below with reference to FIG. 1. FIG. 1 is a block diagram illustrating a configuration of the workout support system 3. The workout support system 3 is a system for supporting a targeted person in a workout, and includes a training apparatus 1 and a workout support apparatus 2, as illustrated. The training apparatus 1 includes a data acquiring section 11 and a training section 12. The workout support apparatus 2 includes a data acquiring section 21 and a generating section 22.

The data acquiring section 11 acquires training data which indicates a workout schedule that is in accordance with a state regarding a workout and that is to be applied in the state.

The training section 12 generates an objective function for generating a workout schedule in accordance with a state, by performing inverse reinforcement learning with use of the training data.

The data acquiring section 21 acquires state data which indicates a state regarding a workout done by the targeted person.

The generating section 22 generates a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state. Note that the objective function used by the generating section 22 may be generated by the training section 12 of the training apparatus 1, or may be generated in another apparatus.

As above, the training apparatus 1 in accordance with the present example embodiment includes: a data acquiring section 11 for acquiring training data which indicates a workout schedule that is in accordance with a state regarding a workout and that is to be applied in the state; and a training section 12 for generating an objective function for generating a workout schedule in accordance with a state, by performing inverse reinforcement learning with use of the training data. Thus, the training apparatus 1 in accordance with the present example embodiment makes it possible to generate an objective function for generating a workout schedule in accordance with a state, and therefore provides an example advantage of making it possible to generate a workout schedule in consideration of a state regarding a workout.

In addition, as described above, the workout support apparatus 2 in accordance with the present example embodiment includes: a data acquiring section 21 for acquiring state data which indicates a state regarding a workout done by the targeted person; and a generating section 22 for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state. Thus, the workout support apparatus 2 in accordance with the present example embodiment provides an example advantage of making it possible to generate a workout schedule in consideration of a state regarding a workout.

### Training Program

The above functions of the training apparatus 1 can be implemented via a program. The training program in accordance with the present example embodiment causes a computer to function as: a data acquiring means for acquiring training data which indicates a workout schedule that is in accordance with a state regarding a workout and that is to be applied in the state; and a training means for generating an objective function for generating a workout schedule in accordance with a state, by performing inverse reinforcement learning with use of the training data. This training program makes it possible to generate an objective function for generating a workout schedule in accordance with a state, and therefore provides an example advantage of making it possible to generate a workout schedule in consideration of a state regarding a workout.

### Workout Support Program

The above functions of the workout support apparatus 2 can be implemented via a program. The workout support program in accordance with the present example embodiment causes a computer to function as: a data acquiring means for acquiring state data which indicates a state regarding a workout done by the targeted person; and a generating means for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state. This workout support program provides an example advantage of making it possible to generate a workout schedule in consideration of a state regarding a workout.

### Flow of Training Method

A flow of a training method in accordance with the present example embodiment will be described below with reference to FIG. 2. FIG. 2 is a flowchart illustrating a flow of the training method. Each of the steps of this training method may be carried out by a processor included in the training apparatus 1, or may be carried out by a processor included in another apparatus. Alternatively, the steps may be carried out by respective processors provided in different apparatuses.

In S11, at least one processor acquires training data which indicates a workout schedule that is in accordance with a state regarding a workout and that is to be applied in the state.

In S12, the at least one processor generates an objective function for generating a workout schedule in accordance with the state, by performing inverse reinforcement learning with use of the training data.

As above, a configuration adopted in the training method in accordance with the present example embodiment is the configuration in which at least one processor acquiring training data which indicates a workout schedule that is in accordance with a state regarding a workout and that is to be applied in the state and the at least one processor generating an objective function for generating a workout schedule in accordance with the state, by performing inverse reinforcement learning with use of the training data are included. Thus, the training method in accordance with the present example embodiment makes it possible to generate an objective function for generating a workout schedule in accordance with a state, and therefore provides an example advantage of making it possible to generate a workout schedule in consideration of a state regarding a workout.

### Flow of Workout Support Method

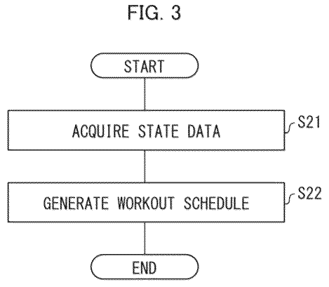

A flow of a workout support method in accordance with the present example embodiment will be described below with reference to FIG. 3. FIG. 3 is a flowchart illustrating a flow of the workout support method. Each of the steps of this workout support method may be carried out by a processor included in the workout support apparatus 2, or may be carried out by a processor included in another apparatus. Alternatively, the steps may be carried out by respective processors provided in different apparatuses.

In S21, at least one processor acquires state data which indicates a state regarding a workout done by a targeted person.

In S22, the at least one processor generates a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

As above, a configuration adopted in the workout support method in accordance with the present example embodiment is the configuration in which at least one processor acquiring state data which indicates a state regarding a workout done by a targeted person and the at least one processor generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state are included. Thus, the workout support method in accordance with the present example embodiment provides an example advantage of making it possible to generate a workout schedule in consideration of a state regarding a workout.

### Second Example Embodiment

### Outline

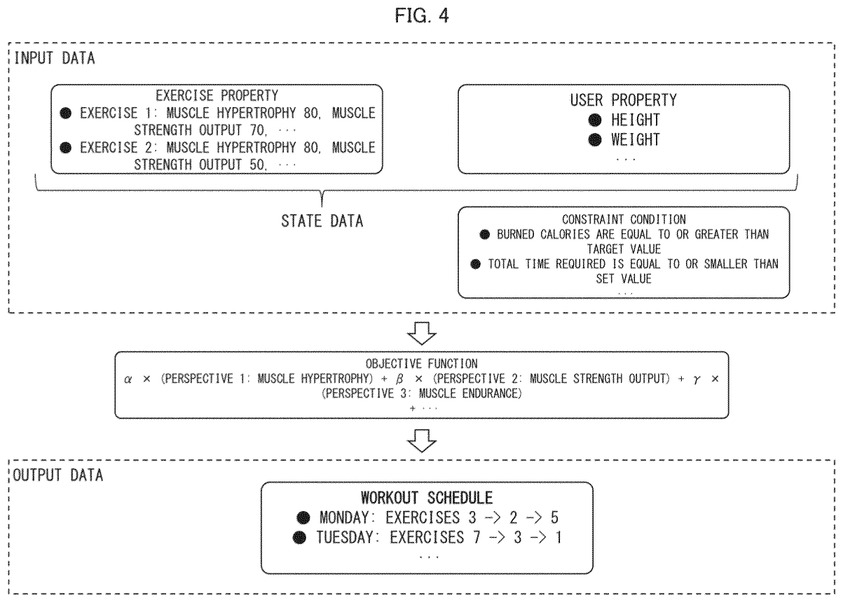

The outline of a workout support method in accordance with the present example embodiment will be described below on the basis of FIG. 4. FIG. 4 is a representation of the outline of the workout support method (hereinafter, referred to as the present method) in accordance with the present example embodiment.

As illustrated, input data in the present method contains: an exercise property which indicates workout exercises from which a targeted person who does a workout can select, and the characteristics of the workout exercises; a user property which indicates a characteristic of the targeted person; and a constraint condition used in generating a workout schedule. Among these properties, the exercise property and the user property are state data which indicates a state regarding a workout done by the targeted person. In the present method, a workout schedule which satisfies the constraint condition and which is in accordance with the state indicated by the state data is generated.

Specifically, the exercise property illustrated in FIG. 4 indicates workout exercises from which the targeted person can select and effects expected to be brought about by each of the workout exercises. For example, the exercise property illustrated in FIG. 4 indicates that the workout exercise “exercise 1” can be selected, and also indicates that this exercise has a muscle hypertrophy effect of 80 and a muscle strength output improvement effect of 70. Note that a method for evaluating the effects of a workout is not particularly limited. Any workout effect evaluated by any evaluation method can be included in the list of exercises. For example, the effect of improving burned calories and muscle endurance may be associated with each exercise. Further, in addition to the effects, a physical activity intensity, a part of the body on which a load is placed, etc. can be included in the exercise property. Note that the burned calories may be calories burned per unit time, or in a case where the amount of workout time of the exercise is determined, the burned calories may be calories burned throughout the entire amount of time.

The user property illustrated in FIG. 4 indicates the height and the weight of the targeted person. The user property only needs to indicate a characteristic of the targeted person, and may be, for example, the maximum workout time in a day, the minimum burned calories in a day, the maximum physical activity intensity in a day, age, gender, occupation, sports experience, and the goal of the workout. The maximum workout time in a day, etc. may be set so as to vary depending on the day of the week.

The constraint condition illustrated in FIG. 4 indicates that the burned calories are equal to or greater than a target value and that the total time required for the workout is equal to or smaller than a set value. Any constraint condition may be set, and for example, a constraint condition of placing a load on the parts of the body thoroughly over a week, or any other constraint condition can be set. Such a constraint condition can be subjected to setting and a change which can freely be made by the targeted person.

In the present method, a workout schedule is generated by performing an optimization calculation with use of the input data as described above and an objective function, the objective function being generated by inverse reinforcement learning with use of training data which indicates a workout schedule that is in accordance with a state regarding a workout and that is to be applied to the input data.

The objective function above contains a weight value which indicates a degree to which importance is put on each of the perspectives used for evaluating a workout schedule. In the objective function illustrated in FIG. 4, a first perspective is “muscle hypertrophy”, a second perspective is “muscle strength output”, and a third perspective is “muscle endurance”, and the respective weight values of these perspectives are α, β, and γ. The perspectives can be automatically determined at the time of training, or may be subjected to setting and a change which can be made by the targeted person. The perspectives to be set only need to be related to a workout schedule. For example, the combinatorial compatibility between workout exercises can be set as a perspective.

Output data, i.e. a workout schedule, illustrated in FIG. 4 indicates, for each day of the week, workout exercises to be done and the order in which the workout exercises are done. For example, the workout schedule illustrated in FIG. 4 indicates that on Monday, a workout is done in the order of exercises 2, 3, and then 5. Note that in the present method, the workout schedule is not limited to the form of the example of FIG. 4, but the workout schedule of any form can be generated. For example, the workout schedule in which the order of exercises is not defined but the combination of the exercises is defined can be generated, or the workout schedule by month can be generated.

### Configuration of Workout Support Apparatus

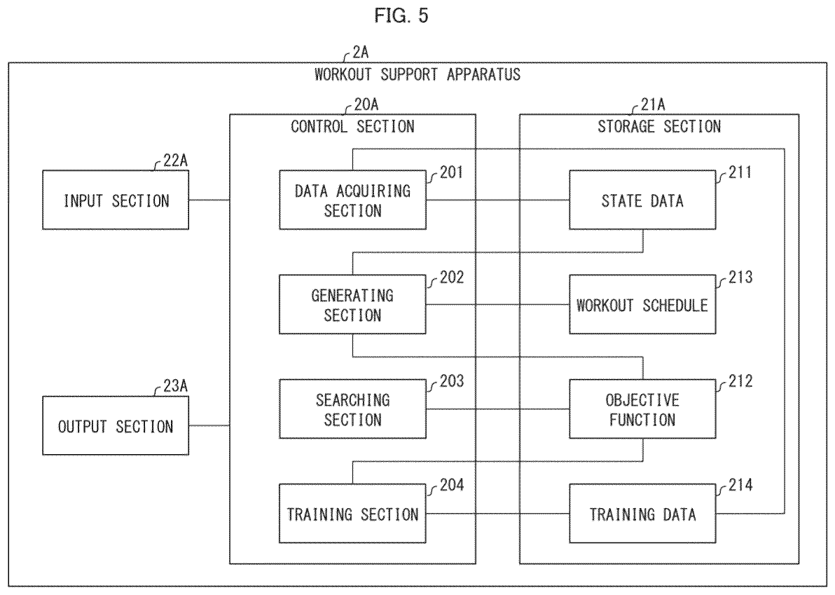

A workout support apparatus 2A in accordance with the present example embodiment will be described below on the basis of FIG. 5. FIG. 5 is a block diagram illustrating an example main configuration of the workout support apparatus 2A. The workout support apparatus 2A generates a workout schedule for a targeted person, to support the targeted person in a workout. Further, the workout support apparatus 2A also has the function of the training apparatus 1 of the first example embodiment, i.e. the function of generating an objective function which contains a weight value indicating a degree to which importance is put on each of the perspectives used for evaluating a workout schedule.

The workout support apparatus 2A includes: a control section 20A for performing overall control of the sections of the workout support apparatus 2A; and a storage section 21A for storing various kinds of data used by the workout support apparatus 2A, as illustrated. The workout support apparatus 2A further includes: an input section 22A for accepting input of various kinds of data to the workout support apparatus 2A; and an output section 23A through which the workout support apparatus 2A outputs various kinds of data. Although an example in which the output section 23A is a display on which various kinds of data are outputted and displayed is described, the output section 23A may output data in another form of output, such as audio output or printed output.

The control section 20A includes: a data acquiring section 201; a generating section 202; a searching section 203; and a training section 204. The storage section 21A has stored therein state data 211, an objective function 212, a workout schedule 213, and training data 214. The searching section 203 will be described later in the section “Display of search keyword”.

The data acquiring section 201 acquires state data 211 which indicates a state regarding a workout done by the targeted person. The state data 211 only needs to indicate a state regarding a workout done by the targeted person. For example, the state data 211 may contain information on a workout itself such as the exercise property illustrated in FIG. 4, or may contain information on the targeted person themselves such as the user property.

The data acquiring section 201 may also acquire a constraint condition used in generating a workout schedule. The data acquiring section 201 acquires training data 214 which indicates a workout schedule that is in accordance with a state regarding a workout and that is to be applied in the state. A method for acquiring these kinds of data is not particularly limited. For example, the data acquiring section 201 may acquire the state data 211, the constraint condition, and the training data 214 which are inputted via the input section 22A.

The generating section 202 generates a workout schedule in accordance with a state indicated by the state data acquired by the data acquiring section 201. More specifically, the generating section 202 generates a workout schedule 213 in accordance with the state indicated by the state data acquired by the data acquiring section 201, by performing an optimization calculation with use of an objective function 212, the objective function 212 being generated by inverse reinforcement learning with use of training data 214 which indicates a workout schedule that is in accordance with a state regarding a workout and that is to be applied in the state. A method for generating the workout schedule 213 will be described later in the section “Optimization calculation”.

The training section 204 uses the training data 214 to generate the objective function 212 used for generating a workout schedule in accordance with a state. As described on the basis of FIG. 4, the objective function 212 generated by the training section 204 not only indicates each of the perspectives used for evaluating a workout schedule but also contains a weight value which indicates a degree to which importance is put on each of the perspectives. A method for generating the objective function 212 will be described later in the section “Objective function training”.

As above, the workout support apparatus 2A in accordance with the present example embodiment includes: a data acquiring section 201 for acquiring state data 211 which indicates a state regarding a workout done by the targeted person; and a generating section 202 for generating a workout schedule 213 according to the state indicated by the state data acquired by the data acquiring section 201, by performing an optimization calculation with use of an objective function 212 generated by inverse reinforcement learning with use of training data 214 which indicates a workout schedule that is in accordance with a state regarding a workout and that is to be applied in the state. Thus, the workout support apparatus 2A in accordance with the present example embodiment provides an example advantage of making it possible to generate a workout schedule in consideration of a state regarding a workout.

As above, in the workout support apparatus 2A in accordance with the present example embodiment, the data acquiring section 201 acquires a constraint condition used in creating a workout schedule for the targeted person, and the generating section 202 generates the workout schedule 213 which satisfies the constraint condition acquired. Thus, the workout support apparatus 2A in accordance with the present example embodiment provides an example advantage of making it possible to generate the workout schedule 213 which satisfies a desired constraint condition, in addition to the example advantage provided by the workout support apparatus 2 in accordance with the first example embodiment.

The workout support apparatus 2A in accordance with the present example embodiment further has the function of a training apparatus. That is, the workout support apparatus 2A includes: a data acquiring section 201 for acquiring training data 214 which indicates a workout schedule that is in accordance with a state regarding a workout and that is to be applied in the state; and a training section 204 for generating an objective function 212 for generating a workout schedule in accordance with a state, by performing inverse reinforcement learning with use of the training data 214. The workout support apparatus 2A in accordance with the present example embodiment makes it possible to generate the objective function 212, which contains a weight value indicating a degree to which importance is put on each of the perspectives used for evaluating a workout schedule, and therefore provides an example advantage of making it possible to generate the workout schedule 213 in consideration of perspectives on which a targeted person who does a workout puts importance.

### Objective Function Training

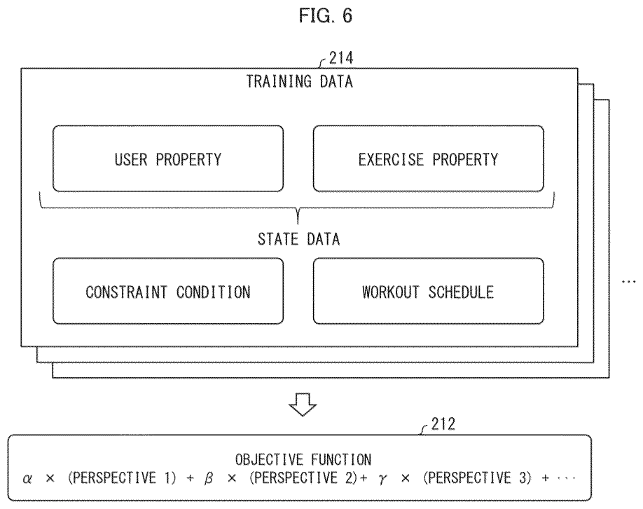

Training, carried out by the training section 204, of the objective function 212 will be described below on the basis of FIG. 6. FIG. 6 is a representation of the outline of training of the objective function 212. The training data 214 illustrated in FIG. 6 contains a user property and an exercise property which are state data, and also contains a constraint condition and a workout schedule.

The training data 214 only needs to indicate a workout schedule that is in accordance with a state regarding a workout and that is to be applied in the state. For example, the training data 214 may indicate a workout schedule implemented by a person (hereinafter, referred to as an expert) who is included in the people having actually done workouts and who has yielded remarkable results. In this case, the user property and the exercise property in the training data 214 are set respectively to the user property of the expert and the property of a workout exercise done by the expert. Further, the constraint condition and the workout schedule of the training data 214 may be set respectively to the constraint condition set at the time of creating the workout schedule for the expert and the workout schedule implemented by the expert.

In the following description, the training data 214 is described in connection with the expert in the present example embodiment. However, the training data 214 is not limited by the expert. The training data 214 only needs to be a combination of data indicating a state regarding a workout and data indicating a workout schedule to be applied in the state.

In addition, the training data 214 does not necessarily need to be generated on the basis of the workout schedule having actually been implemented. For example, training data generated by creating not only typical state data but also a suitable workout schedule which corresponds to the typical state data may be taken as the training data 214. For example, in a case where there is a workout schedule suitable for men in their twenties, training data generated by associating, with this workout schedule, the exercise property of a workout exercise contained in the workout schedule and a user property typical to men in their twenties may be taken as the training data 214. A constraint condition may be contained in the training data 214, if needed.

The training section 204 carries out training with use of the plurality of pieces of training data 214 as described above of respective states different from each other, to generate an objective function 212 which contains a weight value indicating a degree to which importance is put on each of perspectives used for evaluating a workout schedule. It can be said that this training is for learning of the expert's intention in adopting a workout schedule contained in one training data 214 when the expert is in the state indicated in the state data contained in that training data 214. As described above, any perspective can be set.

In the training, first of all, the training section 204 sets each of the weight values of the objective function 212 to an initial value. Next, the generating section 202 generates a workout schedule in accordance with a state indicated by the state data contained in the training data 214, by performing an optimization calculation with use of the objective function 212, the weight values of which are each set to the initial value. The training section 204 then updates the weight values such that the difference between the workout schedule indicated in the training data 214 and the workout schedule generated by the generating section 202 decreases. By repeatedly carrying out these processes until the difference between the workout schedule indicated in the training data 214 and the workout schedule generated by the generating section 202 sufficiently decreases, training of the objective function 212 ends.

As a specific method of the training, various methods used in typical inverse reinforcement learning can also be used. For example, maximum entropy inverse reinforcement learning may be used. In this case, the training section 204 uses the principle of maximum entropy to express a probability distribution of the objective function and approximates the probability distribution of the objective function to the true probability distribution (i.e. maximum likelihood estimation), to train the objective function. It is also possible to determine, by training, perspectives suitable to evaluate a workout schedule contained in the training data 214.

It can be said that the objective function 212 generated by the above-described training indicates the decision-making standard of the expert. For example, the objective function 212 which contains the weight value for the perspective “muscle hypertrophy” greater than that for the perspective “muscle endurance” indicates that considering both of the perspectives of muscle hypertrophy and muscle endurance, the expert has put greater importance on muscle hypertrophy than on muscle endurance to create a workout schedule.

### Optimization Calculation

With use of the objective function 212, it is possible to calculate an evaluation value for evaluating whether a workout schedule is good or bad. Thus, the generating section 202 only needs to generate a workout schedule an evaluation value of which is the maximum, the evaluation value being calculated with use of the objective function 212. A method of solving an optimization problem with use of the objective function, the state data, and the constraint condition is any method. For example, the generating section 202 may use an optimization solver to generate an optimum workout schedule from the objective function 212, the state data 211, and the constraint condition. The generating section 202 can use, as the optimization solver, a common application program, which is, for example, IBM ILOG CPLEX, Gurobi Optimizer, or S CIP.

### Switch Between Objective Functions

In the workout support apparatus 2A in accordance with the present example embodiment, the plurality of objective functions 212 prepared in advance may be stored in the storage section 21A, or the like. In this case, the generating section 202 may use an objective function that is included in the plurality of objective functions 212 prepared in advance and that is in accordance with the targeted person who does a workout, to generate the workout schedule 213. This configuration makes it possible to use an objective function 212 which is included in the plurality of objective functions 212 and which particularly fits the targeted person among the plurality of objective functions 212, and therefore provides an example advantage of making it possible to generate the workout schedule 213 which particularly fits the targeted person, in addition to the example advantage provided by the workout support apparatus 2 in accordance with the first example embodiment.

For example, the plurality of objective functions 212 according to the purpose of a workout may be stored in the storage section 21A or the like. In this case, the generating section 202 can use the objective function 212 according to the purpose of a workout of the targeted person, to generate the workout schedule 213 which matches the purpose of the targeted person.

### Generation of Workout Schedule Containing BGM

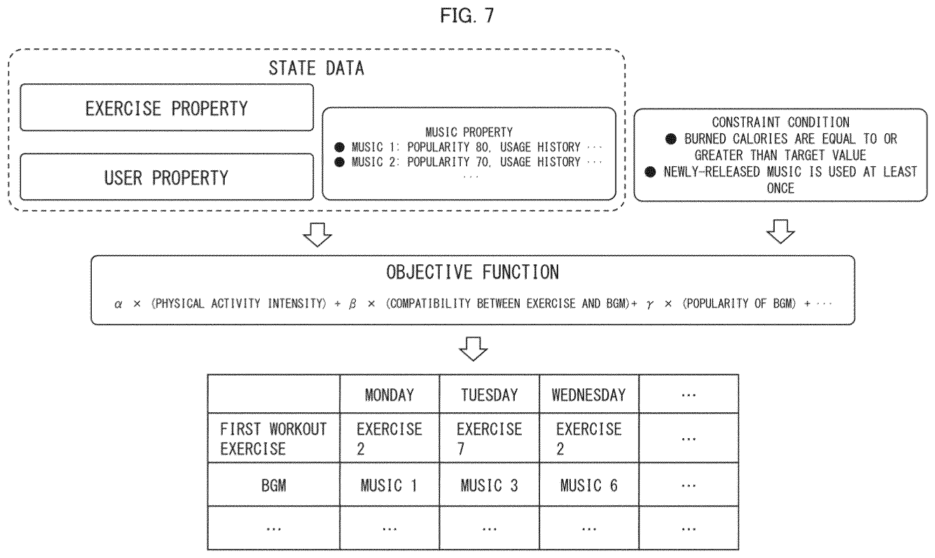

The workout support apparatus 2A is capable of generating a workout schedule which contains back ground music (BGM). This will be described below on the basis of FIG. 7. FIG. 7 is a representation of an example of generating a workout schedule which contains BGM. The example of FIG. 7 differs from the example of FIG. 4: in that the state data contains music property; in the details of the constraint conditions and the objective function; and in that each of the workout exercises is associated with music which serves as BGM in the workout schedule generated.

The music property is data which indicates pieces of music which can be used as BGM and the characteristics of the pieces of music. For example, the music property illustrated in FIG. 7 not only indicates that the music “music 1” can be used as BGM, but also indicates that the popularity of this music is 80 and indicates a usage history of this music as BGM. Note that the music property only needs to indicate pieces of music which can be used as BGM and the characteristics of the pieces of music, and is not limited to the example illustrated in FIG. 7. Examples of the music property may include the title of music, a genre, a release date, an album name, an artist's name, the length of music, loudness, a tune, a tempo, and a meter. Besides these properties, examples of the music property may include: a degree to which the music is suitable for dance; a sense of realism; a degree to which a positive impression is received; the strength of the feeling of being given energy by the music; whether an electronic music instrument is used; whether the music is instrumental music (music without singing); and whether the music is close to speech.

In a case where the workout schedule which contains BGM is generated, a constraint condition regarding BGM can be set in addition to the constraint conditions regarding a workout. The constraint conditions illustrated in FIG. 7 includes a condition where newly-released music is used at least once. By using such a constraint condition, BGM is determined such that newly-released music is definitely contained once at the minimum in each workout. Note that the definition of the newly-released music may be determined in advance. For example, the newly-released music may be defined as music within a half year of the release date. In a case where the workout schedule which contains BGM is generated, the objective function which contains a perspective used in selecting BGM is used. The perspective only needs to be related to BGM. For example, the objective function illustrated in FIG. 7 contains “compatibility between exercise and BGM” and “popularity of BGM”, which are the perspectives used in selecting BGM”, in addition to the “physical activity intensity”, which is the perspective used for evaluation of a workout schedule.

The objective function, as described above, for generating the workout schedule which contains BGM can be generated by training in which the training data 214 is used, the training data 214 indicating: a workout schedule that is in accordance with a state regarding a workout and that is to be applied in the state; and music to be played in the workout. For example, the training data 214 may be used, the training data 214 indicating: the workout schedule having done by the expert and the exercise property; and BGM played by an expert during the implementation of the workout schedule and the music property of the BGM. This makes it possible to generate an objective function which indicates a decision-making standard used in the expert selecting BGM.

The “compatibility between exercise and BGM” can be evaluated with use of, for example, a music usage history. That is, music which is used as BGM many times or frequently in one workout exercise can be evaluated as being compatible with the exercise. In this manner, what property to be used for evaluating a perspective may be determined in advance. This applies to the perspective regarding a workout.

The training section 204 may use any feature selection technique to automatically select a perspective such as the “compatibility between exercise and BGM”. An example feature selection method in inverse reinforcement learning which can be used by the training section 204 is “Teaching Risk”. The feature selection by “Teaching Risk” is to set an ideal parameter in an objective function and compare the ideal parameter with a parameter which is in a training process, to select, as an important feature, a feature (i.e. perspective) which makes the difference between the two parameters smaller.

As a matter of course, a technique for feature selection which can be used by the training section 204 is not limited to “Teaching Risk”. For example, the training section 204 can use the approach disclosed in Patent Application Publication PCT/JP2020/032848, to perform feature selection.

In the example of FIG. 7, a workout schedule which indicates, for each day of the week, a workout exercise and music which serves as BGM of the workout exercise is generated based on the state data, the constraint condition, and the objective function as described above. For example, the workout schedule of FIG. 7 indicates that one of the workout exercises to be done on Monday is “exercise 2”, and BGM to be played during the workout of this exercise is “music 1”.

The workout support apparatus 2A can generate a workout schedule in which a plurality of pieces of music are associated with a single workout exercise. Further, the workout support apparatus 2A can generate a workout schedule in which one or more pieces of music are associated with a plurality of workout exercise done in succession. In presenting a plurality of pieces of music to a targeted person, the workout support apparatus 2A may present the plurality of pieces of music as a play list.

As above, in the workout support apparatus 2A in accordance with the present example embodiment, the generating section 202 uses an objective function 212 having been trained with use of the training data 214 which contains information indicating music played during a workout, to generate the workout schedule 213 which contains music played during a workout. Thus, the workout support apparatus 2A in accordance with the present example embodiment provides an example advantage of making it possible to generate workout schedule 213 which is more appealing, in addition to the example advantage provided by the workout support apparatus 2 in accordance with the first example embodiment.

### Display of Search Keyword

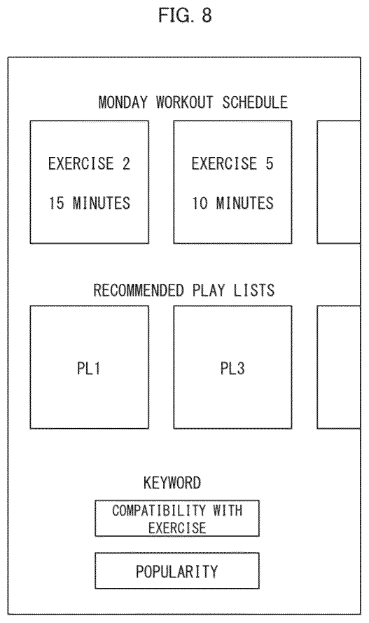

The workout support apparatus 2A may present to a targeted person pieces of music which serve as BGM during a workout, the music being determined as described above, to cause the targeted person to make a final decision of music. This will be described below on the basis of FIG. 8. FIG. 8 is a representation of an example display screen with a workout schedule and BGM.

In the example display screen of FIG. 8, as a Monday workout schedule, two workout exercises “exercise 2” and “exercise 5” and the times required for the workout exercises are indicated. Further, in this example display screen, not only “PL1” is indicated as a recommended play list corresponding to the “exercise 2”, but “PL3” is also indicated as a recommended play list corresponding to the “exercise 5”. These exercises and play lists are determined by the generating section 202 with use of the objective function 212.

The generating section 202 may display workout exercises and recommended play lists for exercises subsequent to “exercise 5” in response to, for example, the operation of scrolling the display screen sideways. Further, the generating section 202 may display workout exercises and recommended play lists for days subsequent to Monday in response to a predetermined operation. Furthermore, the generating section 202 may display each of the pieces of music contained in a play list, or each of the pieces of music contained in a play list may be displayed in response to the operation from the targeted person.

In the example display screen of FIG. 8, in a case of adopting a recommended play list as it is, the targeted person may perform a corresponding operation. Alternatively, the targeted person may select music serving as BGM by themselves, without adopting a recommended play list. The “keyword” in the example display screen of FIG. 8 assists the targeted person in selecting music, and is displayed by the searching section 203.

The searching section 203 displays a word or phrase which indicates a perspective regarding music and which is a search term for searching for music, the perspective being included in the perspectives indicated in the objective function 212 and used for evaluating a workout schedule. The workout support apparatus 2A, which includes the searching section 203, provides an example advantage of making it possible to offer an easy search for music which matches a perspective, in addition to the example advantage provided by the workout support apparatus 2 in accordance with the first example embodiment.

Specifically, in the example display screen of FIG. 8, two keywords which are “compatibility with exercise” and “popularity” are indicated. These keywords are words or phrases which indicate the perspective regarding music included in perspectives which are all indicated in the objective function. In a case where a keyword is selected, the searching section 203 searches the pieces of music which can be selected as BGM for music which matches the keyword, and displays the search result. When desired music is contained in the search result displayed, the targeted person can select the music as BGM to be played during a workout.

The searching section 203 may select the above perspective by utilizing the structure of the objective function. For example, in a case where the weight for the perspective “popularity” is great in the objective function having been trained, the searching section 203 may display the keyword “popularity”.

Assume, for example, that in selecting BGM to be played during “exercise 2”, the keyword “compatibility with exercise” is selected. In this case, the searching section 203 may refer to the music property illustrated in FIG. 7, determine a predetermined number of pieces of music which rank high on the number or frequency of uses as BGM during “exercise 2”, and display the pieces of music as candidates for BGM.

The workout support apparatus 2A may accept registration of favorite music and play list. The workout support apparatus 2A may then display the music and play list having been registered as favorites, together with recommended play lists. This makes it possible to make it easier for a targeted person to set BGM they like.

### Flow of Processes

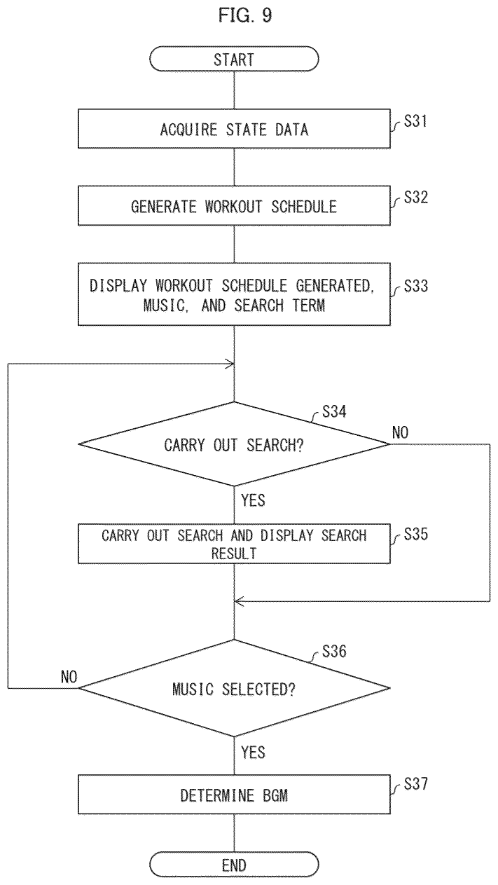

Processes (workout support method) carried out by the workout support apparatus 2A will be described below on the basis of FIG. 9. FIG. 9 is a flowchart illustrating a flow of processes carried out by the workout support apparatus 2A. Note that described below is an example in which a workout schedule that contains music to be played during a workout is generated.

In S31, the data acquiring section 201 acquires the state data 211 which indicates a state regarding a workout done by a targeted person. In S31, the data acquiring section 201 may also acquire a constraint condition used in generating a workout schedule.

In S32, the generating section 202 generates the workout schedule 213 in accordance with the state illustrated in the state data 211 acquired in S31. Specifically, the generating section 202 generates the workout schedule 213 by performing an optimization calculation with use of an objective function 212, the objective function 212 being generated by inverse reinforcement learning with use of the training data 214 which indicates a workout schedule that is in accordance with a state regarding a workout and that is to be applied to the state. This workout schedule 213 contains music to be played during a workout.

In S33, the generating section 202 causes the workout schedule 213 generated in S32 and music to be played during the implementation of the workout schedule 213 to be outputted and displayed on the output section 23A. Further, the searching section 203 causes a search term for searching for music to be outputted and displayed on the output section 23A. As described above, the search term displayed by the searching section 203 is a word or phrase which indicates a perspective regarding music included in the perspectives indicated in the objective function 212 and used for evaluating a workout schedule. Note that the music may be displayed by the piece, or a plurality of pieces of music may be collectively displayed as a play list (see FIG. 8).

In S34, the searching section 203 judges whether to carry out a search. For example, in a case of detecting the operation of selecting from among the displayed keywords, the searching section 203 may judge that a search should be carried out. In a case where the judgment is YES in S34, the method continues to the process of S35, and in a case where the judgment is NO in S34, the method continues to the process of S36.

In S35, the searching section 203 searches for music with the keyword selected by the targeted person from among the keywords displayed in S33, and outputs and displays the search result on the output section 23A. Note that the searching section 203 may search for music with use of a keyword inputted by the targeted person or a narrowing criterion selected by the targeted person.

In S36, the searching section 203 judges whether music serving as BGM has been selected. The music to be selected may be music displayed in S33, or may be music displayed in S35. Further, the selection of music may be accepted via the input section 22A. In a case where the judgment is YES in S36, the method continues to S37, and in a case where the judgment is NO in S37, the method returns to S34.

In S37, the generating section 202 determines that the music selected in S36 is the BGM to be played during a workout. The workout schedule 213 which contains music to be played during a workout is thus completed, and the processes of FIG. 9 end.

### Variation

A performer which carries out each of the processes described in the example embodiments above is any performer, and is not limited to the above examples. For example, it is possible to construct a workout support system having the same functions as the workout support apparatus 2A, with use of a plurality of apparatuses capable of communicating with each other. For example, it is possible to construct the workout support system having the same functions as the workout support apparatus 2A, by dispersedly providing, in a plurality of apparatuses, the respective blocks illustrated in FIG. 5.

### Software Implementation Example

Some or all of the functions of each of the training apparatus 1 and the workout support apparatuses 2 and 2A may be implemented by hardware such as an integrated circuit (IC chip), or may be implemented by software.

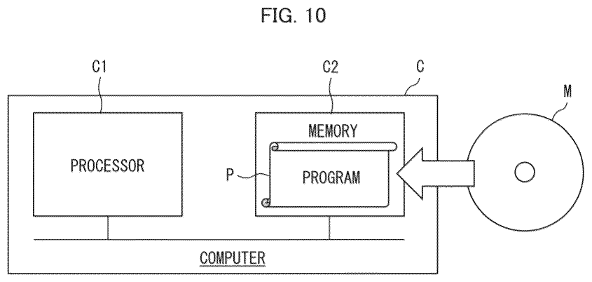

In the latter case, the training apparatus 1 and the workout support apparatuses 2 and 2A are provided by, for example, a computer that executes instructions of a program that is software for implementing the foregoing functions. An example (hereinafter, computer C) of such a computer is illustrated in FIG. 10. The computer C includes at least one processor C1 and at least one memory C2. The memory C2 has recorded thereon a program P for causing the computer C to operate as the training apparatus 1 and the workout support apparatuses 2 and 2A. The processor C1 of the computer C retrieves the program P from the memory C2 and executes the program P, so that the functions of the training apparatus 1 and the workout support apparatuses 2 and 2A are implemented.

Examples of the at least one processor C1 can include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 can include a flash memory, a hard disk drive (HDD), a solid state drive (SSD), and a combination thereof.

The computer C may further include a random access memory (RAM) into which the program P is loaded at the time of execution and in which various kinds of data are temporarily stored. The computer C may further include a communication interface via which data is transmitted to and received from another apparatus. The computer C may further include an input-output interface via which input-output equipment such as a keyboard, a mouse, a display or a printer is connected.

The program P can be recorded on a non-transitory tangible recording medium M capable of being read by the computer C. Examples of such a recording medium M can include a tape, a disk, a card, a semiconductor memory, and a programmable logic circuit. The computer C can obtain the program P via such a recording medium M. The program P can be transmitted via a transmission medium. Examples of such a transmission medium can include a communication network and a broadcast wave. The computer C can also obtain the program P via such a transmission medium.

### Additional Remark 1

The present invention is not limited to the above example embodiments, but may be altered in various ways by a skilled person within the scope of the claims. For example, the present invention also encompasses, in its technical scope, any example embodiment derived by appropriately combining technical means disclosed in the above example embodiments.

### Additional Remark 2

The whole or part of the example embodiments disclosed above can be described as, but not limited to, the following supplementary notes.

### Supplementary Note 1

A workout support apparatus including: a data acquiring means for acquiring state data which indicates a state regarding a workout done by a targeted person; and a generating means for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

### Supplementary Note 2

The workout support apparatus described in supplementary note 1, in which the data acquiring means is configured to acquire a constraint condition used in generating the workout schedule for the targeted person, and the generating means is configured to generate the workout schedule that satisfies the constraint condition.

### Supplementary Note 3

The workout support apparatus described in supplementary note 1 or 2, in which the generating means is configured to use an objective function that is included in a plurality of objective functions prepared in advance each of which is the objective function and that is in accordance with the targeted person, to generate the workout schedule.

### Supplementary Note 4

The workout support apparatus described in any one of supplementary notes 1 to 3, in which the training data contains information which indicates music to be played during a workout, and the generating means is configured to generate the workout schedule that contains music to be played during a workout.

### Supplementary Note 5

The workout support apparatus described in supplementary note 4, further including a searching means for displaying a word or phrase which indicates a perspective regarding music and which is a search term for searching for music, the perspective being included in perspectives indicated in the objective function and used for evaluating a workout schedule.

### Supplementary Note 6

A workout support method including:
at least one processor acquiring state data which indicates a state regarding a workout done by a targeted person; and the at least one processor generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

### Supplementary Note 7

A workout support program for causing a computer to function as: a data acquiring means for acquiring state data which indicates a state regarding a workout done by a targeted person; and a generating means for generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

### Supplementary Note 8

A training apparatus including: a data acquiring means for acquiring training data which indicates a workout schedule that is in accordance with a state regarding a workout and that is to be applied in the state; and a training means for generating an objective function for generating a workout schedule in accordance with a state, by performing inverse reinforcement learning with use of the training data.

### Supplementary Note 9

A training method including: at least one processor acquiring training data which indicates a workout schedule that is in accordance with a state regarding a workout and that is to be applied in the state; and the at least one processor generating an objective function for generating a workout schedule in accordance with a state, by performing inverse reinforcement learning with use of the training data.

### Supplementary Note 10

A training program for causing a computer to function as:
a data acquiring means for acquiring training data which indicates a workout schedule that is in accordance with a state regarding a workout and that is to be applied in the state; and a training means for generating an objective function for generating a workout schedule in accordance with a state, by performing inverse reinforcement learning with use of the training data.

### Additional Remark 3

The whole or part of the example embodiments disclosed above can be described as follows.

A workout support apparatus including at least one processor, the at least one processor carrying out: a data acquiring process of acquiring state data which indicates a state regarding a workout done by a targeted person; and a generating process of generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

This workout support apparatus may further include a memory, and the memory may have stored therein a program for causing the at least one processor to carry out the data acquiring process and the generating process. A computer-readable non-transitory tangible recording medium may have this program recorded thereon.

A training apparatus including at least one processor, the at least one processor carrying out: a data acquiring process of acquiring training data which indicates a workout schedule that is in accordance with a state regarding a workout and that is to be applied in the state; and a training process of generating an objective function for generating a workout schedule in accordance with a state, by performing inverse reinforcement learning with use of the training data.

This training apparatus may further include a memory, and this memory may have stored therein a program for causing the at least one processor to carry out the data acquiring process the and training process. A computer-readable non-transitory tangible recording medium may have this program recorded thereon.

### REFERENCE SIGNS LIST

1: Training apparatus
2, 2A: Workout support apparatus
11, 21, 201: Data acquiring section
12, 204: Training section
22, 202: Generating section
203: Searching section

What is claimed is:

1. A workout support apparatus comprising at least one processor, the at least one processor carrying out: a data acquiring process of acquiring state data which indicates a state regarding a workout done by a targeted person; and
a generating process of generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

2. The workout support apparatus according to claim 1, wherein in the data acquiring process, the at least one processor acquires a constraint condition used in generating the workout schedule for the targeted person, and in the generating process, the at least one processor generates the workout schedule that satisfies the constraint condition.

3. The workout support apparatus according to claim 1, wherein in the generating process, the at least one processor uses an objective function that is included in a plurality of objective functions prepared in advance each of which is the objective function and that is in accordance with the targeted person, to generate the workout schedule.

4. The workout support apparatus according to claim 1, wherein the training data contains information which indicates music to be played during a workout, and
in the generating process, the at least one processor generates the workout schedule that contains music to be played during a workout.

5. The workout support apparatus according to claim 4, the at least one processor further carries out a searching process of displaying a word or phrase which indicates a perspective regarding music and which is a search term for searching for music, the perspective being included in perspectives indicated in the objective function and used for evaluating a workout schedule.

6. A workout support method comprising:
at least one processor acquiring state data which indicates a state regarding a workout done by a targeted person; and
the at least one processor generating a workout schedule in accordance with the state indicated by the state data, by performing an optimization calculation with use of an objective function, the objective function being generated by inverse reinforcement learning with use of training data which indicates a workout schedule that is in accordance with a state regarding a workout and that is to be applied in the state.

7. A non-transitory storage medium storing a workout support program for causing a computer to function as a workout support apparatus recited in claim 1, the program causing the computer to carry out the data acquiring process and the generating process.

8. A training apparatus comprising at least one processor, the at least one processor carrying out: a data acquiring process of acquiring training data which indicates a workout schedule that is in accordance with a state regarding a workout and that is to be applied in the state; and
a training process of generating an objective function for generating a workout schedule in accordance with a state, by performing inverse reinforcement learning with use of the training data.

9. (canceled)

10. A non-transitory storage medium storing a_training program for causing a computer to function as a training apparatus recited in claim 8, the program causing the computer to carry out the data acquiring process and the training process.
